SEQUENCE LISTING (4 sequences)

SEQ ID NO: 1 - DNA, 37 bp, Artificial Sequence
  Description of Artificial Sequence Synthetic primer
caccggatcctgtcgcgagactcgcaagagacagcag

SEQ ID NO: 2 - DNA, 36 bp, Artificial Sequence
  Description of Artificial Sequence Synthetic primer
cccgaattcgcacgtcttcccgtcgaggatgagctc

SEQ ID NO: 3 - DNA, 25 bp, Artificial Sequence
  Description of Artificial Sequence Synthetic primer
caccggatcctgtggaccctacacc

SEQ ID NO: 4 - DNA, 27 bp, Artificial Sequence
  Description of Artificial Sequence Synthetic primer
cccgaattcgcacgtcttcccgtcgag

(19) United States
(12) Patent Application Publication
(10) Pub. No.: US 2008/0280296 A1
(43) Pub. Date: Nov. 13, 2008

(54) METHOD FOR DETECTION OF FOOT-AND-MOUTH DISEASE VIRUS WITH CHROMATOGRAPHIC STRIP TEST

(76) Inventors: Tsu-Han CHEN, Taipei (TW); Chu-Hsiang Pan, Taipei (TW); Ming-Hwa Jong, Taipei (TW)

Correspondence Address: RABIN & Berdo, PC, 1101 14TH STREET, NW, SUITE 500, WASHINGTON, DC, 20005, US

(21) Appl. No.: 11/956,562

(22) Filed: Dec. 14, 2007

(30) Foreign Application Priority Data
May 11, 2007 (TW) 096116924

(51) Int. Cl. C12Q 1/68

(52) U.S. Cl. 435/6

(57) ABSTRACT

The present invention discloses a method for detection of foot-and-mouth disease virus with chromatographic strip test. Firstly, the nucleic acid sequence of FMDV NSPs is set up, the nucleic acid sequence is amplified by the reverse transcriptase polymerase chain reaction (RT-PCR) method, the recombinant vector is constructed and performed through a prokaryotic system to transform and express the recombinant protein, and the purified recombinant protein is mass produced. Design principles of the method are based on immunoassay and chromatographic analysis. The advantages are easy and simple to handle, no need of elaborate equipment, only one drop of body fluid is required to quickly complete the qualitative test in 10-20 minutes, and operating with a portable POCT (Point of care testing) instrument to complete the quantitative detection within 40-50 minutes.

### BACKGROUND OF THE INVENTION

1. Field of the Invention

The invention relates to a clinical immunology and detection for antibodies against structural and/or nonstructural proteins of foot-and-mouth disease virus. More particularly, the invention relates to a rapid, qualitative and quantitative method for detection of foot-and-mouth disease virus with chromatographic strip test with both of high sensitivity and specificity.

2. Description of Related Art

Foot and mouth disease (FMD) is a highly contagious disease of cloven-hoofed animals, the economic animal that infected notably bovine, pig, and sheep. FMD is characterized by fever, vesicular lesions, and erosion of the epithelium of the mouth, tongue, nares, muzzle, feet, and teats. Foot-and-mouth disease virus (FMDV) is a positive stranded RNA virus belonging to the Aphthovirus genus in the family Picornaviridae, which is a small nonenveloped virus with an ˜8.5 k bp genome which codes for structural as well as nonstructural proteins (NSPs). There are seven serotypes, known as serotypes O, A, C, Asia 1, SAT 1, SAT 2, and SAT 3, recognized worldwide and each of them has no cross protection. This disease is still not effectively differential from swine vesicular disease (SVD), Vesicular stomatitis (VS), Vesicular exanthema (VE), and San Miguel sea lion virus in clinical diagnosis.

In 1997, a devastating outbreak of FMD in Taiwan was caused by a serotype O virus (referred to here as O/TAW/97) with an atypical porcinophilic phenotype. By studies show that an altered nonstructural protein, 3A (condons 93 to 102; hereafter referred to as the 93-102 deletion), is a primary determinant of restricted growth on bovine cells in vitro and significantly contributes to bovine attenuation of O/YUN/TAW/97 in vivo. The outbreak had a severe impact on the national economy due to costs of control and trade restrictions (estimated at over 6 billion U.S. dollars) in 1997. Another strain that had a full-length 3A coding region were identified bovine-virulent virus (O/TAW/2/99) isolated from a sub-clinically infected animal on Taiwanese island of kinmen. This FMD virus, O/TAW//2/99, is a topotype of South Asia serotype O and invaded Taiwan from 1999. When an outbreak of this disease occurs, quarantine measures are applied and the animals on the infected farm are culled and their carcasses destroyed to break the chain of infection as quickly as possible. When considered necessary, preventive culling of animals in suspect farms may also be applied. Routine vaccination is used widely and successfully to control FMD in countries where the virus is endemic or poses recurrent threats of virus incursions from neighboring countries. Intensive vaccination of livestock over decades eventually allows such countries to reduce the incidence of FMD to the point at which they are able to eradicate the infection, allowing them to acquire disease-free status. Eradication programmes in Taiwan include systematic vaccination accompanied by large sero-surveys through NSP antibody testing to ensure the absence of residual viral activity.

During FMDV replication, antibodies are produced against both viral capsid proteins and non-structural proteins (NSPs). The latter proteins are involved in the replication of the virus. Most FMDV vaccines that are used globally in routine vaccination are inactivated whole-virus vaccines grown in cell culture, all FMD vaccines require a concentration process in their production, manufacturers are encouraged to include a purification process for completely removing NSP and therefore animals vaccinated against FMD will develop antibodies to structural proteins only. Purification of vaccine antigens serves two purposes; the elimination of proteins that can induce allergic reactions and secondly, NSPs are removed or their concentration considerably reduced. Therefore, it is expected that vaccines prepared from purified antigen will not induce antibodies against NSPs. This has allowed for the development of tests for the detection of antibodies against NSPs having the potential to differentiate vaccinated from infected animals with the added advantage of detecting antibodies independent of virus serotype. Considerable effort and attention is now being directed toward the development of new methods and techniques for the rapid and accurate detection of anti-NSP antibodies, and harmonization and standardization of current diagnostic techniques. Several diagnostic tests, such as latex beads agglutination test and enzyme-linked immunosorbent assay (ELISA), to detect NSPs (2C, 3A, 3B, 3AB, 3ABC, 3D) have so far been the most successful in distinguishing infected animals from those that have been vaccinated. This virus specific NSP has been produced either in recombinant Escherichia coli or in insect cells infected with the appropriate recombinant Baculovirus and peptide synthesis. In order to reduce the number of false positives, an enzyme-linked immunoelectrotransfer blot assay (EITB) has been used as a confirmatory test. EITB uses several NSPs (2C, 3A, 3B, 3ABC, 3D and etc.) and all the test and analysis should be performed in laboratory and accomplished with various instruments, equipments and professional techniques.

World Organization for Animal Health (OIE) had announced Taiwan is the vaccinal foot and mouth disease-free status on May 22, 2003. All cloven-hoofed livestock have so far been inoculated inactivated FMD vaccines. Owing to inactivated FMD vaccines will not induce antibodies against NSPs which is induced by naturally infection, Enzyme-linked Immunosorbent Assay (ELISA) is often combined with commercial products, UBI (United Biochemical Inc., Hauppauge, N.Y., USA), Ceditest (Ceditest® FMDV-NS, Cedi Diagnostics B.V., Lelystad, The Netherlans) and Chekit (IDEXX Laboratories Inc., Westbrook, Me., USA), to detect serum antibodies against FMDV NSPs for differentiating vaccinated animals from natural infected ones. However, the process of commercial ELISA kits is inconvenient and consumes the time, and the long time of cell culture of commercial ELISA kits lead to extend the time of diagnosis. Owing to FMDV is high contagious infection, any delay will make epidemic situation become serious and cause more damage of production and economy.

### SUMMARY OF THE INVENTION

Accordingly, in order to promote the detection efficiency of commercial ELISA kits, the present invention provides a method for detection of foot-and-mouth disease virus with chromatographic strip test, wherein the nucleic acid sequence of FMDV NSPs is set on a test strip, the nucleic acid sequence is amplified by the reverse transcriptase polymerase chain reaction (RT-PCR) method, the recombinant vector is constructed and performed through a prokaryotic system to transform and express the recombinant protein, and the purified recombinant protein is mass produced. By that to provide a rapid and one step method for detecting the antibodies and/or antigens in liquid samples collected from doubtful-infected animals. Moreover, the method diagnoses whether the animals are infected with FMDV or not by permitting to provide a rapid protection of infected but vaccinated animals. The advantage is easy and simple to handle, no need of elaborate equipment and only one drop of body fluid is required and the qualitative test can be completed quickly in 10-20 minutes. Another purpose of the present invention is to get the method for detection of foot-and-mouth disease virus with chromatographic strip test operating with a portable POCT (Point of care testing) instrument which is viewed as a quantitative detection for antibodies of FMDV NSPs in body fluid, and the quantitative detection can be completed within 40-50 minutes. Using NSPs as antigen substances has the benefit that it can quantity produce the functional and soluble recombinant protein, have security with no alive virus, easily purify and obtain high concentration protein. And then the manufacturing processing is stable, the procedure is standardized and harmonized and the cost is reduced. Hence, the present invention produces a rapid, simple, sensitive and stable product with both of specificity and accuracy. It is expected when the foot-and-mouth disease vaccine inoculation execution is suspended in Taiwan that means Taiwan is assented into the non-epidemic country, the present invention can provide a checking method to quarantine unit. Moreover, it is a new aim in the future that the chromatographic test strip (pen-side strip) of detection for antibodies of FMDV NSPs can also be popularized to the international market. The chromatographic test strip (pen-side strip) is suitable for epidemic prevention workers at quarantinable area. It is particularly a prompt and ideal tool for routine disease examination.

As the above, the method of the present invention comprises the following process:
(1) Building a target gene of non-structure protein nuclei acid sequence of FMDV O/TAW/97 and O/TAW/99;
(2) The RT-PCR method was utilized to design a specific primer and prepare the cDNA templates of target pathogen and DNA purified product;
(3) The recombinant fragment made of (2) is constructed on a pET vector;
(4) Checking the sequencing result of nucleic acid sequence to conform the insert test of recombinant gene;
(5) Performing the transformation, induced expressing the recombinant protein and purifying; and
(6) Performing the functional test of the recombinant protein.

Finally, a chromatographic test strip of the present invention is made following the above process, evaluated and compared with the three types of ELISA kits of FMD non-structure protein antibodies.

In the present invention, the chromatographic test strip of FMD non-structure protein antibodies is applied in clinical quarantine for qualitative decision, diagnosis and quantitative decision. The properties of the present invention possess the advantages such as: (a) sensitivity; (b) specificity; (c) simplification; (d) stability; and (c) economy. These advantages are described as following:

(a) Sensitivity: the chromatographic test strip of the present invention can detect the positive body fluid which was diluted 10⁻⁶ fold.

(b) Specificity: the chromatographic test strip of the present invention is confirmed that it can simultaneously detect antibodies to the non-structure proteins of four serotypes of FMDV O, A, C and Asia 1.

(c) Simplification: the detection steps of chromatographic test strip of the present invention are simpler than the commercial ELISA kits, there is no cleaning step in the reaction, and the test strip can rapidly accomplish the detection with diluted rough sample. It is easier than the ELISA kits which generally need 4-5 hour reaction time. The test strip can perform the qualitative and quantitative analysis. The qualitative analysis can be completed in 10-20 minutes and the quantitative analysis can be completed in 40-50 minutes. In the quantitative analysis, there is no need of expensive desktop equipment and the result is rapidly obtained by operating with POCT detector.

(d) Stability: the preservation of the chromatographic test strip of the present invention is over the half year at the room temp. 26° C.˜30° C.

(c) Economy: the chromatographic test strip of the present invention is easy to be quantity produced to get the cost down.

The present invention is to develop a set of method for detection of foot-and-mouth disease virus with chromatographic strip test and the advanced technology like genetic engineering is used for producing the chromatographic test trip (pen-side strip). It is based on the design principle of safety consideration and there is no risk of doubting the reactivation of pathogen. It is novel that only one drop of body fluid is required and the qualitative test can be completed quickly in 10-20 minutes. In addition, operating with a portable pocket type POCT instrument researched and developed by Taiwan Unison Biotech Company, the quantitative analysis can be completed within 50 minutes. It is expected when the foot-and-mouth disease vaccine inoculation execution is suspended in Taiwan, the present invention can provide a checking method to quarantine unit to assist swineherd in rapidly knowing whether the sick pig is infected with pathogen or not. Furthermore, the pen-side test strip can also be applied in and popularized to overseas countries that are infected with foot-and-mouth disease. Hence, a new model of disease diagnosis can be established and in the future, even this technology can be researched and developed to widely apply in other animal diseases. This pen-side strip will be a prompt and ideal tool that can be provided to Taiwan and overseas infected area for quarantine technician to conduct routine disease examination. Hence, the present invention possesses characteristics of usefulness and marketing.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is the flow chart of method for detection of foot-and-mouth disease virus with chromatographic strip test according to one of the preferred embodiments of the present invention.

FIG. 2 is a positive result illustration of chromatographic test strip according to one of the preferred embodiments of the present invention.

FIG. 3 is a negative result illustration of chromatographic test strip according to one of the preferred embodiments of the present invention.

Table 1 is a comparative statement of tested value according to the comparison of the chromatographic test strip (pen-side strip) with the three types of commercial ELISA kits in the preferred embodiments of the present invention.

### DETAILED DESCRIPTION OF THE PREFERRED EMBODIMENTS

Reference will now be made in detail to the preferred embodiments of the invention, examples of which are illustrated in the accompanying drawings.

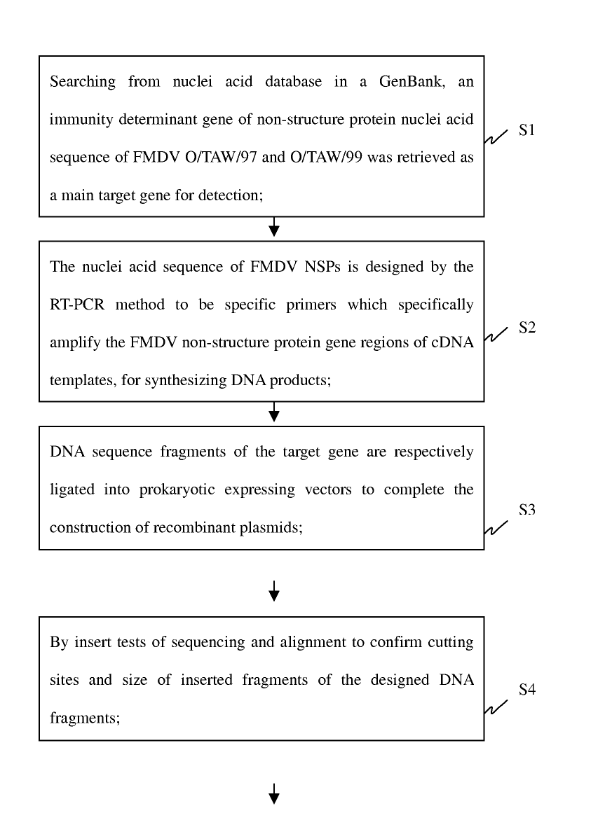

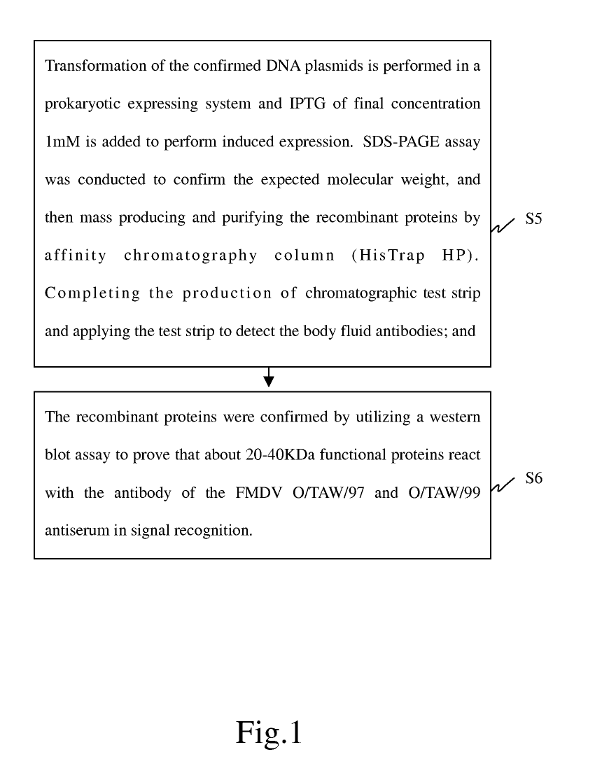

FIG. 1 is the flow chart of method for detection of foot-and-mouth disease virus with chromatographic strip test according to one of the preferred embodiments of the present invention. Referring to FIG. 1, the method of the present invention comprises the following process:

Step S1: Searching from nuclei acid database in a GenBank, an immunity determinant gene of non-structure protein nuclei acid sequence of FMDV O/TAW/97 and O/TAW/99 was retrieved as the main target gene for detection.

Step S2: The above non-structure protein nuclei acid sequence of FMDV is designed by the RT-PCR method to be specific primers which specifically amplify the FMDV non-structure protein gene regions of cDNA templates, wherein

forward primer(FMDV-3ABC-F):
5′-CACCGGATCCTGTCGCGAGACTCGCAAGAGACAGCAG-3′;
reverse primer (FMDV-3ABC-R):
5′-CCCGAATTCGCACGTCTTCCCGTCGAGGATGAGCTC-3′;
forward primer (FMDV-3BC-F):
5′-CACCGGATCCTGTGGACCCTACACC -3′;
reverse primer (FMDV-3BC-R):
5′-CCCGAATTCGCACGTCTTCCCGTCGAG -3′

for synthesis of DNA products.

Step S3: DNA sequence fragments of the target gene are respectively ligated into pET vectors to complete the construction of recombinant plasmids (pTH525 B and pTH294B).

Step S4: By insert tests of sequencing and alignment to confirm cutting sites (BamHI, EcoRI) and the size of inserted fragments of the designed DNA fragments (525 bp, 294 bp).

Step S5: Performing the transformation of confirmed DNA plasmid in a prokaryotic expressing system, cloning the colony grown in LB (Luria-Bertani) cell culture and generate till 0.8˜1 of OD600, and adding IPTG (Isopropylthiogalactoside) of final concentration 1 mM to perform induced expression at 37□, 250 rpm. At this time, the inserted gene DE3 in E. coli BL21 (DE3) generates RNA polymerase T7 which is an enzyme. This enzyme promotes the promoter T7 on the pET vector to express the recombinant genes. The 12% SDS-PAGE assay was conducted to confirm the expected molecular weight of redissolved recombinant protein. And then mass producing and purifying the recombinant proteins by HisTrap HP affinity chromatography column (Amersham Biosciences). Completing the production of chromatographic test strip and applying the test strip to detect the body fluid antibodies.

Step S6: The recombinant proteins were confirmed by utilizing a western blot assay to prove that about 20-40 KDa functional proteins will react with the antibody of the FMDV O/TAW/97 and O/TAW/99 antiserum in signal recognition.

The estimation of detection result of chromatographic test strip is described as following:

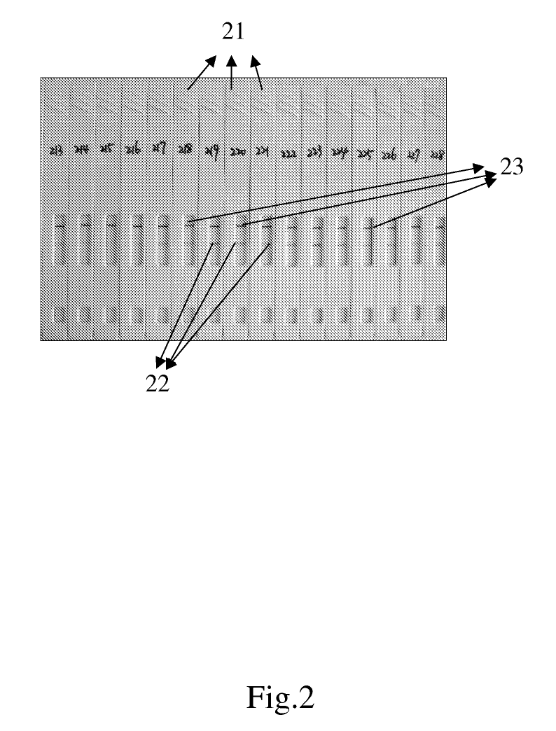

FIG. 2 is a positive result illustration of chromatographic test strip according to one of the preferred embodiments of the present invention. As shown in FIG. 2, each of the chromatographic test strips 21 was reacted with a body fluid such as a whole blood or serum and appeared the positive result. There are two obvious bands on each of the chromatographic test strips 21. One band is appeared on the test site (T) 22 and the other band is appeared on the control site (C) 23.

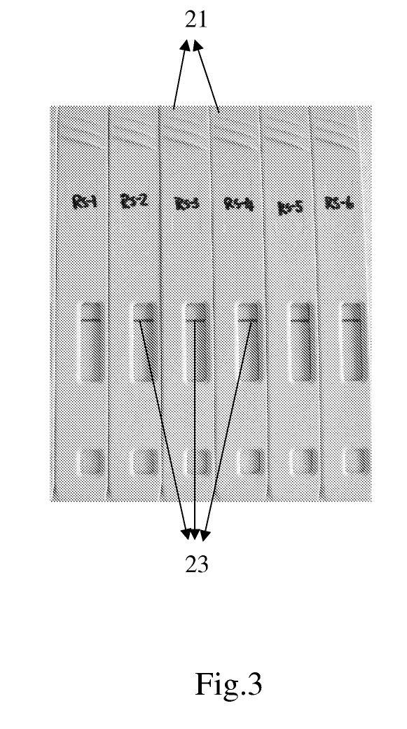

FIG. 3 is a negative result illustration of chromatographic test strip according to one of the preferred embodiments of the present invention. As shown in FIG. 3, each of the chromatographic test strips 21 was reacted with a body fluid and appeared the negative result. There is only one obvious band on the control site (C) 23 of the chromatographic test strip.

Finally, evaluating the comparison of the chromatographic test strip (pen-side strip) with the three types of ELISA kits of FMD non-structure protein antibodies, the chromatographic test strip of the present invention is made following the above process, tested and compared with the three types of commercial ELISA kits of FMD non-structure protein antibodies. From the comparison results between pen-side strips and the three kinds of commercial ELISA kits, it is discovered that the pen-side strips can check out earlier than the three types of commercial ELISA kits, work without the expensive equipment and rapidly obtain the test result. Table 1 is a comparative statement of tested value according to the comparison of the chromatographic test strip (pen-side strip) with the three types of commercial ELISA kits in the preferred embodiments of the present invention. As shown in Table 1, ELISA kit A is CEDITEST-FMD-3ABC ELISA (Ceditest® FMDV-NS, Cedi Diagnostics B.V., Lelystad, The Netherlans), ELISA kit B is UBI-FMD-3B ELISA (United Biochemical Inc., Hauppauge, N.Y., USA) and ELISA kit C is CHEKIT-FMD-3ABC ELISA (IDEXX Laboratories Inc., Westbrook, Me., USA). The test time of all the three types of commercial ELISA kits is 4˜5 hours. The Specificity of ELISA kit-A is 100%. The Specificity of ELISA kit-B is 85.3˜100%. The Specificity of ELISA kit-C is 100%. The test time of the chromatographic test strip is the minimum and the qualitative test can be completed quickly in 10-20 minutes. Operating with the POCT detector, the quantitative analysis can be completed within 40-50 minutes. If the FMD is break out again, the decrease of the test time can help the epidemic prevention workers to control the disaster of quarantinable area. Moreover, the sensitivity and specificity of the pen-side strip can respectively reach 93.3˜95.6% and 98.8˜100%, which are equivalent to that of the three commercial ELISA kits. And, no need of expensive desktop equipment could further the convenience for epidemic prevention workers testing and proceeding with working. The method is based on solid state chromatographic analysis and combined with immune colloidal metal and improved materials. Comparing with the present invention, the process of commercial ELISA kits is inconvenient and consumes the time. The long time of cell culture of commercial ELISA kits lead to extend the time of diagnosis. Finally, the pen-side strip of the present invention is confirmed that it can simultaneously detect antibodies to non-structure proteins of four serotypes of FMDV 0, A, C and Asia 1, and is not react the antibodies to swine vesicular disease virus (SVDV).

TABLE 1

| | Test time | Specificity^{a} | Sensitivity^{a} | Equipment |
|---|---|---|---|---|
| Chromato- | Qualitative | 98.8~100% | 93.3~95.6% | Quantitative |
| graphic test | test: | | | test with |
| strip | 10-20 min.; | | | POCT |
| | Quantitative | | | detector |
| | test: | | | |
| | 40-50 min.; | | | |
| ELISA kit-A | 4-5 hr. | 100% | 96.7~98.1% | desktop |
| | | | | equipment |
| ELISA kit-B | 4-5 hr. | 85.3~100% | 97.5~100% | desktop |
| | | | | equipment |
| ELISA kit-C | 4-5 hr. | 100% | 45.6~46.7% | desktop |
| | | | | equipment |

In the above, the primers are FMDV-3ABC-F and FMDV-3ABC-R; FMDV-3BC-F and FMDV-3BC-R; non-structure proteins are protein G and/or protein A; structure and non-structure proteins comprise at least one of VP1, VP2, VP3, VP4, Lb, 2B, 2C, 3A, 3D, 3AB, 3BC or 3ABC; In the method for detection of foot-and-mouth disease virus with chromatographic strip test, the FMDV antibodies particularly use the FMDV non-structure proteins comprising at least one of Lb, 2B, 2C, 3A, 3AB, 3BC, 3ABC or 3D.

The reverse transcriptase polymerase chain reaction (RT-PCR) method of the Step S2 is described in detail as following: Firstly, the nucleic acid is prepared from the FMDV-O type collected from the cell culture, 100 μL is took in the 1.5 mL microtube, 1 mL Trizol reagent is added in, solution is oscillated 30 seconds and stayed at room temp. 5 min., 0.2 mL chloroform is added in, mixtured well and stayed at room temp. 3 min., centrifugation 15 min. at 12000 rpm, 4° C., extract the supernatant liquid, mixtured with isometric isopropanol and stayed at room temp. 10 min., centrifugation 20 min. at 12000 rpm, 4° C., remove the supernatant liquid and through the vacuum centrifugal dry, 100 μL distilled water is added in, which is deal twice with the DEPC. Utilizing the PT-PCT method, the nucleic acid solution is respectively dispensed into 0.5 mL microtubes, then add in 10 μL of 10 fold Dynazyne buffer solution, 0.02 micromole (mM) base dNTP (dATP, dCTP, dGTP, dTTP respectively is 0.5 mM), 8 units RNasin, 2 units AMV reverse transcriptase, 1 units Supertherm polymerase and 0.01 nanomole primer, finally add the DEPC water till the total volume is 50 μL. After mixturing well, the solution is stayed in a heat cycle machine (Applied Biosystems, Gene Amp PCR system 2400) for preheat reverse transcription with one cycle (working 40 min. at 42° C. and then working 50 sec. at 94° C.), and proceed with the PCT reaction whose condition is denature 30 sec. at 94° C., refine 30 sec. at 55° C., elongate 1 min. at 68° C., working 35 cycles, 7 min. at 72° C. till cool down at 4° C. All the RT-PCT products made of the above process are electrophoretic analyzed with 2% Agarose gel.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A method for detection of foot-and-mouth disease virus with chromatographic strip test, wherein primers are designed by the nuclei acid sequence of non-structure proteins (NSPs) of foot-and-mouth disease virus (FMDV), a reverse transcriptase polymerase chain reaction (RT-PCR) method is utilized to amplify the nuclei acid of virus, recombinant vectors are constructed and performed through a prokaryotic system to transform and express recombinant proteins, and the purified recombinant proteins are mass produced; a chromatographic test strip (pen-side strip) is made following the above process, only one drop of body fluid is required to the test strip for completing qualitative test, and the test strip is operated with a portable POCT (Point of care testing) detector for completing quantitative test; and the method comprises the following process:
(S1) Searching from nuclei acid database in a GenBank, an immunity determinant gene of non-structure protein nuclei acid sequence of FMDV O/TAW/97 and O/TAW/99 was retrieved as a main target gene for detection;
(S2) The nuclei acid sequence of FMDV NSPs is designed by the RT-PCR method to be specific primers which specifically amplify the FMDV non-structure protein gene regions of cDNA templates, for synthesizing DNA products;
(S3) DNA sequence fragments of the target gene are respectively ligated into prokaryotic expressing vectors to complete the construction of recombinant plasmids;
(S4) By insert tests of sequencing and alignment to confirm cutting sites and size of inserted fragments of the designed DNA fragments;
(S5) Transformation of the confirmed DNA plasmids is performed in a prokaryotic expressing system and IPTG (Isopropylthiogalactoside) of final concentration 1 mM is added to perform induced expression. SDS-PAGE assay was conducted to confirm the expected molecular weight, and then mass producing and purifying the recombinant proteins by affinity chromatography column (HisTrap HP). Completing the production of chromatographic test strip and applying the test strip to detect the body fluid antibodies; and
(S6) The recombinant proteins were confirmed by utilizing a western blot assay to prove that about 20-40 KDa functional proteins react with the antibody of the FMDV O/TAW/97 and O/TAW/99 antiserum in signal recognition.

2. The method according to claim 1, wherein principles of the design are based on immunoassay and chromatographic analysis.

3. The method according to claim 1, wherein the specific primers are forward primers FMDV-3ABC-F and FMDV-3BC-F.

4. The method according to claim 3, wherein the FMDV-3ABC-F is 5′-CACCGGATCCTGTCGCGAGACTCGCAAGAGACAGCAG-3′ (SEQ ID NO: 1), and the FMDV-3BC-F is 5′-ACCGGATCCTGTGGACCCTACACC-3′ (SEQ ID NO: 3).

5. The method according to claim 1, wherein the specific primers are reverse primers FMDV-3ABC-R and FMDV-3BC-R.

6. The method according to claim 5, wherein the FMDV-3ABC-R is 5′-CCCGAATTCGCACGTCTTCCCGTCGAGGATGAGCTC-3′ (SEQ ID NO: 2) and the FMDV-3BC-R is 5′-CCCGAATTCGCACGTCTTCCCGTCGAG-3′ (SEQ ID NO: 4).

7. The method according to claim 1, wherein structure and non-structure proteins of FMDV comprise at least one of VP1, VP2, VP3, VP4, Lb, 2B, 2C, 3A, 3D, 3AB, 3BC or 3ABC.

8. The method according to claim 1, wherein the non-structure proteins are protein G and/or protein A.

9. The method according to claim 1, wherein the FMDV antibodies particularly use the FMDV non-structure proteins comprising at least one of Lb, 2B, 2C, 3A, 3AB, 3BC, 3ABC or 3D.

10. The method according to claim 1, wherein the chromatographic test strip simultaneously detect antibodies to the non-structure proteins of four serotypes of FMDV O, A, C and Asia 1.

11. The method according to claim 1, wherein the body fluid is a whole blood or serum.

12. The method according to claim 1, wherein the chromatographic test strip completes the qualitative test within 10-20 minutes.

13. The method according to claim 1, wherein portable POCT detector completes the quantitative test within 40-50 minutes.
